(19) United States
(12) Patent Application Publication
(10) Pub. No.: US 2025/0191675 A1
(43) Pub. Date: Jun. 12, 2025

(54) MACHINE-LEARNING TECHNIQUES IN PROTEIN DESIGN FOR VACCINE GENERATION

(71) Applicant: Sanofi Pasteur, Inc., Swiftwater, PA (US)

(72) Inventors: Philip Davidson, Cambridge, MA (US); Maryann Giel-Moloney, Cambridge, MA (US); Konstantin Zeldovich, Cambridge, MA (US)

(21) Appl. No.: 18/845,368

(22) PCT Filed: Mar. 10, 2023
(86) PCT No.: PCT/US2023/014962

§ 371 Date: Sep. 9, 2024

Related U.S. Application Data
(60) Provisional application No. 63/319,700, filed on Mar. 14, 2022.
(60) Provisional application No. 63/319,692, filed on Mar. 14, 2022.

(51) Int. Cl. G16B 15/30; G16B 40/20

(52) U.S. Cl. CPC G16B 15/30; G16B 40/20

(57) ABSTRACT

A discrete-data object is received and may include a plurality of first discrete values, the discrete-data object may include one or more amino acid sequences. The discrete-data object is converted into a continuous-data object that may include a plurality of first continuous values. To the continuous-data object, a continuous-data algorithm is applied to generate a continuous-result object that may include a plurality of second continuous values. The continuous-result object is converted into a discrete-result object which may include a plurality of second discrete values. A vaccine is manufactured which may include at least one of the group that may include i) a protein defined by the discrete-result object, ii) a nucleic acid capable of producing the protein defined by the discrete-result object, and iii) a delivery vehicle capable of producing the protein defined by the discrete-result object.

### CROSS REFERENCE TO RELATED APPLICATIONS

This application claims priority to U.S. Provisional Application No. 63/319,700, filed on Mar. 14, 2022 and U.S. Provisional Application No. 63/319,692, filed on Mar. 14, 2022, the entire contents of which are incorporated herein by reference.

### TECHNICAL FIELD

This application is related to use of machine learning techniques in the design of vaccines.

### BACKGROUND

Machine learning (IL) is the use of computer algorithms that can improve automatically through experience and by the use of data. It is seen as a part of artificial intelligence. Machine learning algorithms build a model based on sample data, known as training data, in order to make predictions or decisions without being explicitly programmed to do so. Machine learning algorithms are used in a wide variety of applications, such as in medicine, email filtering, speech recognition, and computer vision, where it is difficult or unfeasible to develop conventional algorithms to perform the needed tasks.

A vaccine is a biological preparation that provides acquired immunity to a particular infectious disease. A vaccine typically contains an agent that resembles a disease-causing microorganism and is often made from weakened or killed forms of the microbe, its toxins, or one of its surface proteins. The agent stimulates the body's immune system to recognize the agent as a threat, destroy it, and to further recognize and destroy any of the microorganisms associated with that agent that it may encounter in the future. Vaccines can be prophylactic (to prevent or ameliorate the effects of a future infection by a natural or “wild” pathogen), or therapeutic (to fight a disease that has already occurred, such as cancer). Some vaccines offer full sterilizing immunity, in which infection is prevented completely.

### SUMMARY

The strains used in seasonal influenza vaccines are currently and near-universally chosen by public health authorities. These selections are made yearly, based on observations of immune response in animal models and human studies. However, H3N2 vaccines using the strains recommended by the public health authorities have not been sufficient to elicit broad protection in the general population, e.g., over the past 5 years (2015-2020). Further, during this time-frame public data shows that immunological relatedness has split into divergent clades wherein each clade is protective to itself while protection against other clades can be limited. The present disclosure provides a solution to this problem. The implementations described in this disclosure provide for an algorithm that introduces mutations into a given starting strain and uses a differentiable machine learning approach such that a separate model predicts that the modified antigen will be highly protective against both the homologous as well as heterologous clades. In an example experiment, the algorithm was used to optimize the HA1 sequence of H3 hemagglutinins (positions 16 to 345) and then wildtype signal peptide and HA2 regions were grafted on to create a complete hemagglutinin sequence. An exemplary modified antigen sequence starting from A/Singapore/INFIMH-16-0019/2016 is provided with mutated residues indicated in bold:

(SEQ ID NO: 1)
MKTIIALSYILCLVFAQKIPGNDNSTATLCLGHHAVPNGTIVKTITNDR
IEVTNATELVQNSSIGEICDSPHQILDGENCTLIDALLGDPQCDGFQNK
KWDLFVERSKAYSNCYPYDVPDYASLRSLVASSGTLEFNNESFNWTGVT
QNGTSSACIRGSSSSFFSRLNWLTHLNYTYPALNVTMPNKEQFDKLYIW
GVHHPGTDKDQISLYARSSGRITVSTKRSQQAVIPNIGSRPRIRDIPSR
ISIYWTIVKPGDILLINSTGNLIAPRGYFKIRSGKSSIMRSDAPIGKCK
SECITPNGSIPNDKPFQNVNRITYGACPRYVKHSTLKLATGMRNVPEKQ
TRGIFGAIAGFIENGWEGMVDGWYGFRHQNSEGRGQAADLKSTQAAIDQ
INGKLNRLIGKTNEKFHQIEKEFSEVEGRVQDLEKYVEDTKIDLWSYNA
ELLVALENQHTIDLTDSEMNKLFEKTKKQLRENAEDMGNGCFKIYHKCD
NACIGSIRNETYDHNVYRDEALNNRFQIKGVELKSGYKDWILWISFAIS
CFLLCVALLGFIMWACQKGNIRCNICI

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method for manufacturing a vaccine by using a continuous-data algorithm. The method includes receiving a discrete-data object that may include a plurality of first discrete values, the discrete-data object may include one or more amino acid sequences. The method also includes converting the discrete-data object into a continuous-data object that may include a plurality of first continuous values. The method also includes applying, to the continuous-data object, a continuous-data algorithm to generate a continuous-result object that may include a plurality of second continuous values. The method also includes converting the continuous-result object into a discrete-result object that may include a plurality of second discrete values. The method also includes manufacturing a vaccine that may include at least one of the group may include of i) a protein defined by the discrete-result object, ii) a nucleic acid capable of producing the protein defined by the discrete-result object, and a iii) delivery vehicle capable of producing the protein defined by the discrete-result object. Other embodiments of this aspect include corresponding computer systems, apparatus, and computer programs recorded on one or more computer storage devices, each configured to perform the actions of the methods.

Implementations may include one or more of the following features. The method where the one or more amino acid sequences may include: a first amino acid sequence and a second amino acid sequence, each of the first and the second amino acid sequences including respective single letters or respective letter strings. Converting the discrete-data object into the continuous-data object may include: generating, for each first discrete value, a weight-vector of weight values, each weight value representing a likelihood that the first discrete value represents a particular amino acid; generating, for each weight value of each weight-vector, a property-vector of property values, each property value representing a physiochemical property of a particular amino acid; and combining the weight-vector and the property-vector to create the first continuous values of the continuous-data object. Each weight-vector has twenty weight values, each weight value corresponding to one of twenty possible amino acids. Converting the continuous-result object into the discrete-result object may include determining, for each second continuous value, a respective single amino acid, where the determined single amino acids form the plurality of second discrete values. The method further may include: generating a plurality of candidate discrete-result objects; and excluding, from the plurality of candidate discrete-result objects, at least one discrete-result object that specifies an amino acid failing a manufacturability test. Applying the continuous-data algorithm to generate the continuous-result object may include applying a gradient descent with a loss function that determines a loss-value based on a plurality of loss criteria, the loss function may include: a first loss criteria based on an immunological response given two amino acid sequences; a second loss criteria that modifies the loss-value for sub-sequences not found in a dataset of wildtype sequences or sub-sequences not predicted to fold correctly; and a third loss criteria that, for each weight-vector, modifies the loss-value based on the greatest value in the second continuous values. The vaccine is for one of the group that may include of i) influenza, ii) human rhinovirus, iii) hiv and iiiv) a coronavirus disease. Implementations of the described techniques may include hardware, a method or process, or computer software on a computer-accessible medium.

One general aspect includes a system for generating amino acid sequences, the system may include computer memory. The system also includes one or more processors. The system also includes computer-memory storing instructions that, when executed by the processors, cause the processors to perform operations that may include: receiving a discrete-data object comprising a plurality of first discrete values, the discrete-data object comprising one or more amino acid sequences; converting the discrete-data object into a continuous-data object comprising a plurality of first continuous values; applying, to the continuous-data object, a continuous-data algorithm to generate a continuous-result object comprising a plurality of second continuous values; converting the continuous-result object into a discrete-result object comprising a plurality of second discrete values; and manufacturing a vaccine comprising at least one of the group consisting of i) a protein defined by the discrete-result object, ii) a nucleic acid capable of producing the protein defined by the discrete-result object, and iii) a delivery vehicle capable of producing the protein defined by the discrete-result object. Other embodiments of this aspect include corresponding computer systems, apparatus, and computer programs recorded on one or more computer storage devices, each configured to perform the actions of the methods.

Implementations may include one or more of the following features. The system where the one or more amino acid sequences may include: a first amino acid sequence and a second amino acid sequence, each of the first and the second amino acid sequences including respective single letters or respective letter strings. Converting the discrete-data object into the continuous-data object may include: generating, for each first discrete value, a weight-vector of weight values, each weight value representing a likelihood that the first discrete value represents a particular amino acid; generating, for each weight value of each weight-vector, a property-vector of property values, each property value representing a physiochemical property of a particular amino acid; and combining the weight-vector and the property-vector to create the first continuous values of the continuous-data object. Each weight-vector has twenty weight values, each weight value corresponding to one of twenty possible amino acids. Converting the continuous-result object into the discrete-result object may include determining, for each second continuous value, a respective single amino acid, where the determined single amino acids form the plurality of second discrete values. The operations further may include: generating a plurality of candidate discrete-result objects; and excluding, from the plurality of candidate discrete-result objects, at least one discrete-result object that specifies an amino acid failing a manufacturability test. Applying the continuous-data algorithm to generate the continuous-result object may include applying a gradient descent with a loss function that determines a loss-value based on a plurality of loss criteria, the loss function may include: a first loss criteria based on an immunological response given two amino acid sequences; a second loss criteria that modifies the loss-value for sub-sequences not found in a dataset of wildtype sequences or sub-sequences not predicted to fold correctly; and a third loss criteria that, for each weight-vector, modifies the loss-value based on the greatest value in the second continuous values. Implementations of the described techniques may include hardware, a method or process, or computer software on a computer-accessible medium.

One general aspect includes a non-transitory, computer readable media storing instructions that, when executed by one or more processors, cause the one or more processors to perform operations that may include: receiving a discrete-data object comprising a plurality of first discrete values, the discrete-data object comprising one or more amino acid sequences; converting the discrete-data object into a continuous-data object comprising a plurality of first continuous values; applying, to the continuous-data object, a continuous-data algorithm to generate a continuous-result object comprising a plurality of second continuous values; converting the continuous-result object into a discrete-result object comprising a plurality of second discrete values; and manufacturing a vaccine comprising at least one of the group consisting of i) a protein defined by the discrete-result object, ii) a nucleic acid capable of producing the protein defined by the discrete-result object, and iii) a delivery vehicle capable of producing the protein defined by the discrete-result object. Other embodiments of this aspect include corresponding computer systems, apparatus, and computer programs recorded on one or more computer storage devices, each configured to perform the actions of the methods.

Implementations may include one or more of the following features. The media where the one or more amino acid sequences may include: a first amino acid sequence and a second amino acid sequence, each of the first and the second amino acid sequences including respective single letters or respective letter strings. Converting the discrete-data object into the continuous-data object may include: generating, for each first discrete value, a weight-vector of weight values, each weight value representing a likelihood that the first discrete value represents a particular amino acid; generating, for each weight value of each weight-vector, a property-vector of property values, each property value representing a physiochemical property of a particular amino acid; and combining the weight-vector and the property-vector to create the first continuous values of the continuous-data object. Each weight-vector has twenty weight values, each weight value corresponding to one of twenty possible amino acids. Converting the continuous-result object into the discrete-result object may include determining, for each second continuous value, a respective single amino acid, where the determined single amino acids form the plurality of second discrete values. Implementations of the described techniques may include hardware, a method or process, or computer software on a computer-accessible medium.

Also disclosed herein are vaccine compositions comprising a plurality of any of the generated amino acid sequences of the methods described herein.

Also disclosed are vectors, fusion proteins, and cells comprising one or more of the peptides and/or proteins produced according to the methods described herein.

Also disclosed herein are methods of eliciting an immune response in a subject that include administering one or more of the isolated nucleic acids, peptides and/or proteins described herein, thereby eliciting an immune response in the subject.

In one aspect, disclosed herein are methods of inhibiting a viral infection that includes administering to a subject any of the one or more isolated nucleic acids, peptides and/or proteins described herein or any of the vaccines comprising any of the isolated nucleic acids, peptides and/or proteins described herein.

Also disclosed herein are methods of immunizing a subject against influenza virus comprising administering to the subject an immunologically effective amount of the vaccine composition as disclosed herein. Also disclosed herein is a vaccine composition as disclosed herein for use in a method of immunizing a subject against a virus (e.g., an influenza virus). Also disclosed herein is a vaccine composition as disclosed herein for the manufacture of a medicament for use in a method of immunizing a subject against a virus (e.g., an influenza virus). In certain embodiments, the method prevents a viral infection (e.g., an influenza virus infection) in a subject, and in certain embodiments, the method raises a protective immune response (e.g., an HA antibody response and/or an NA antibody response), in the subject. In certain embodiments, the subject is human, and in certain embodiments, the vaccine composition is administered intramuscularly, intradermally, subcutaneously, intravenously, or intraperitoneally.

Another aspect of the disclosure is directed to a method of reducing one or more symptoms of a viral infection (e.g., an influenza virus infection), the method comprising administering to a subject a prophylactically effective amount of the vaccine composition disclosed herein. Also disclosed herein is a vaccine composition as disclosed herein for use in a method of reducing one or more symptoms of a viral infection (e.g., an influenza virus infection). Also disclosed herein is a vaccine composition as disclosed herein for the manufacture of a medicament for use in a method of reducing one or more symptoms of an infection (e.g., an influenza virus infection).

In various embodiments, the methods and compositions disclosed herein treat or prevent disease caused by either or both a seasonal or a pandemic viral strain (e.g., a seasonal or pandemic influenza strain).

In certain embodiments of the methods disclosed herein wherein the subject is human, the human is 6 months of age or older, less than 18 years of age, at least 6 months of age and less than 18 years of age, at least 18 years of age and less than 65 years of age, at least 6 months of age and less than 5 years of age, at least 5 years of age and less than 65 years of age, at least 60 years of age, or at least 65 years of age. For example, the subject is 6 months, 8 months, 10 months, 12 months, 14 months, 16 months, 18 months, 20 months, 22 months, 24 months, 3 years, 4 years, 5 years, 6 years, 10 years, 12 years, 15 years 18 years, 20 years, 21 years, 25 years, 30 years, 35 years, 40 years, 50 years, 60 years, 70 years, 75 years, 80 years, 85 years, or 90 years old. In certain embodiments, the methods disclosed herein comprise administering to the subject two doses of the vaccine composition with an interval of 2-6 weeks, such as an interval of 4 weeks.

Implementations can include any, all, or none of the following features.

The implementations discussed in this disclosure can provide one or more of the following advantages. The implementations can be used to generate hemagglutinin sequences with potential to induce broad protection from influenza infection following vaccination. Notably, the implementations can be used to produce antigens that have a greater than expected recovery rate of functional influenza virus with designed hemagglutinin sequences. These antigens are believed to have broad protection, greater than current standard of care antigens in an animal model. The implementations can be used to generate broadly protective hemagglutinin proteins for use as influenza vaccine antigens, or define sequences of a nucleic acid, or any other delivery vehicle including viral or bacterial vectors, whereby such nucleic acid or delivery vehicle produces the protein for use as influenza vaccine antigen.

By converting discrete-only domain data (e.g., amino acid sequences) into continuous datasets, algorithms designed for continuous data can be used with the discrete data. For example, off-the-shelf solvers, computational maximizers, classifiers, etc. can be applied to amino acid sequences when those tools would not normally be able to operate on the amino acid sequences directly. This can advantageously allow for vaccine development using amino acid sequences and continuous-only algorithms. As such, a machine-learning predictor can be used to predict a mammalian immune response given two protein sequences. For example, an algorithm such as gradient descent can be used on protein sequences targeting an increase in immune response even though such a gradient descent is not normally able to operate on the kind of discrete data that is used to represent protein sequences. Gradient descent can be used to optimize predicted immune response, immunogenicity, and biophysical stability of candidate proteins. Candidate proteins generated with the gradient descent can then be analyzed to determine their efficacy, for example, as a vaccine against a disease caused by diverse or rapidly evolving pathogen strains.

Another advantage of the techniques provided in the present disclosure is to improve likelihood of generating protein sequence data for proteins that can actually exist and be manufactured. As will be understood, it is possible to describe protein sequences that, due to geometry, physical forces, etc., cannot exist. Processes described in this document can be advantageously constrained to only those known to or expected to be manufacturable.

Other features, aspects and potential advantages will be apparent from the accompanying description and figures.

### DESCRIPTION OF DRAWINGS

FIG. 1 is a block diagram of an example system that can be used to manufacture a vaccine.

FIG. 2 is a schematic diagram of data that can be used in the manufacture of a vaccine.

FIGS. 3-6 are flowcharts of example processes that can be used to apply continuous algorithms to discrete data, such as may be used in the manufacture of a vaccine.

FIG. 7 is a swimlane diagram of an example process to manufacture a vaccine.

FIG. 8 is a schematic diagram that shows an example of a computing device and a mobile computing device.

Like reference symbols in the various drawings indicate like elements.

### DETAILED DESCRIPTION

This document describes vaccine creation through machine learning processes. The vaccine creation uses candidate proteins that are generated by a computational process that includes machine learning. An initial antigen sequence is modified using the machine learning techniques into one or more candidate sequence that may be used as a vaccine. However, many machine-learning operations use continuous values, while antigen sequences are often characterized with discrete values. In order to perform continuous-value operations on a dataset of discrete values, the present disclosure provides techniques to transfer the discrete values into continuous values, operate on the continuous values, and then transform the continuous values back into discrete values. In order to produce useful results, these operations can be constrained so that the output of discrete values does not define antigen sequences known to be or expected to be physically impossible.

Influenza virus is a member of the Orthomyxoviridae family. There are three subtypes of influenza viruses: influenza A, influenza B, and influenza C. Influenza A viruses infect a wide variety of birds and mammals, including humans, chickens, ferrets, pigs, and horses. In mammals, most influenza A viruses cause mild localized infections of the respiratory and intestinal tract.

The influenza virion contains a negative-sense RNA genome, which encodes the following nine proteins: hemagglutinin (HA), matrix (M1), proton ion-channel protein (M2), neuraminidase (NA), nonstructural protein 2 (NS2), nucleoprotein (NP), polymerase acidic protein (PA), polymerase basic protein 1 (PB1), and polymerase basic protein 2 (PB2). The HA, M1, M2, and NA are membrane associated proteins, whereas NP, NS2, PA, PB1, and PB2 are nucleocapsid associated proteins. The M1 protein is the most abundant protein in influenza particles. The HA and NA proteins are envelope glycoproteins, which are responsible for virus attachment and cellular entry. The HA and NA proteins are the source of the major immunodominant epitopes for virus neutralization and protective immunity. The HA and NA proteins are considered the most important components for prophylactic influenza vaccines.

HA is a viral surface glycoprotein that generally comprises approximately 560 amino acids and representing 25% of the total virus protein.

NA is a membrane glycoprotein of the influenza viruses. NA is 413 amino acid in length, and is encoded by a gene of 1413 nucleotides. Nine different NA subtypes have been identified in influenza viruses (N1, N2, N3, N4, N5, N6, N7, N8 and N9), all of which have been found among wild birds.

The influenza virus' ability to cause widespread disease stems from its ability to evade the immune system by undergoing antigenic change.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

Adjuvant: As used herein, the term “adjuvant” refers to a substance or combination of substances that may be used to enhance an immune response to an antigen component of a vaccine.

Antigen: As used herein, the term “antigen” refers to an agent that elicits an immune response; and/or (ii) an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism; alternatively or additionally, in some embodiments, an antigen elicits a cellular response (e.g., involving T-cells whose receptors specifically interact with the antigen) in an organism. It will be appreciated by those skilled in the art that a particular antigen may elicit an immune response in one or several members of a target organism (e.g., mice, ferrets, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity, including those induced by heterologous immunogens. Antigens include the NA and HA forms as described herein.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Epitope: As used herein, the term “epitope” includes any moiety that is specifically recognized by an immunoglobulin (e.g., antibody or receptor) binding component in whole or in part. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

Excipient: As used herein, the term “excipient” refers to a non-therapeutic agent that may be included in a pharmaceutical composition, for example to provide or contribute to a desired consistency or stabilizing effect. Suitable pharmaceutical excipients include, for example, starch, glucose, lactose, sucrose, sorbitol, gelatin, malt, rice, flour, chalk, silica gel, sodium stearate, glycerol monostearate, talc, sodium chloride, dried skim milk, glycerol, propylene, glycol, water, ethanol and the like.

Immune response: As used herein, the term “immune response” refers to a response of a cell of the immune system, such as a B cell, T cell, dendritic cell, macrophage or polymorphonucleocyte, to a stimulus such as an antigen, immunogen, or vaccine. An immune response can include any cell of the body involved in a host defense response, including for example, an epithelial cell that secretes an interferon or a cytokine. An immune response includes, but is not limited to, an innate and/or adaptive immune response. As used herein, a protective immune response refers to an immune response that protects a subject from infection (prevents infection or prevents the development of disease associated with infection) or reduces the symptoms of infection. Methods of measuring immune responses are well known in the art and include, for example, measuring proliferation and/or activity of lymphocytes (such as B or T cells), secretion of cytokines or chemokines, inflammation, antibody production and the like. An antibody response or humoral response is an immune response in which antibodies are produced. A “cellular immune response” is one mediated by T cells and/or other white blood cells.

Immunogen: As used herein, the term “immunogen” or “immunogenic” refers to a compound, composition, or substance which is capable, under appropriate conditions, of stimulating an immune response, such as the production of antibodies or a T cell response in an animal, including compositions that are injected or absorbed into an animal. As used herein, “immunize” means to render a subject protected from an infectious disease.

Immunologically effective amount: As used herein, the term “immunologically effective amount” means an amount sufficient to immunize a subject.

Prevention: The term “prevention”, as used herein, refers to prophylaxis, avoidance of disease manifestation, a delay of onset, and/or reduction in frequency and/or severity of one or more symptoms of a particular disease, disorder or condition (e.g., infection for example with influenza virus). In some embodiments, prevention is assessed on a population basis such that an agent is considered to “prevent” a particular disease, disorder or condition if a statistically significant decrease in the development, frequency, and/or intensity of one or more symptoms of the disease, disorder or condition is observed in a population susceptible to the disease, disorder, or condition.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. “Sequence identity” between two nucleic acid sequences indicates the percentage of nucleotides that are identical between the sequences. “Sequence identity” between two amino acid sequences indicates the percentage of amino acids that are identical between the sequences. Homologs or variants of a given gene or protein will possess a relatively high degree of sequence identity when aligned using standard methods.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional and/or structural property of said given sequence, e.g., and in some instances, are functionally and/or structurally equivalent to said given sequence. In some embodiments, a nucleic acid sequence or amino acid sequence having a particular degree of identity to a given nucleic acid sequence or amino acid sequence is functionally and/or structurally equivalent to said given sequence.

Subject: As used herein, the term “subject” means any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In some embodiments, the non-human subject is a mammal (e.g., a rodent, a mouse, a rat, a rabbit, a ferret, a monkey, a dog, a cat, a sheep, cattle, a primate, and/or a pig). In some embodiments, a subject may be a transgenic animal, genetically-engineered animal, and/or a clone. In some embodiments, the subject is an adult, an adolescent or an infant. In some embodiments, terms “individual” or “patient” are used and are intended to be interchangeable with “subject.”

Vaccination: As used herein, the term “vaccination” or “vaccinate” refers to the administration of a composition to generate an immune response, for example to a disease-causing agent such as an influenza virus. Vaccination can be administered before, during, and/or after exposure to a disease-causing agent, and/or to the development of one or more symptoms, and in some embodiments, before, during, and/or shortly after exposure to the agent. Vaccines may elicit both prophylactic (preventative) and therapeutic responses. Methods of administration vary according to the vaccine, but may include inoculation, ingestion, inhalation or other forms of administration. Inoculations can be delivered by any of a number of routes, including parenteral, such as intravenous, subcutaneous, intraperitoneal, intradermal, or intramuscular. Vaccines may be administered with an adjuvant to boost the immune response. In some embodiments, vaccination includes multiple administrations, appropriately spaced in time, of a vaccinating composition.

Vaccine Efficacy: As used herein, the term “vaccine efficacy” or “vaccine effectiveness” refers to a measurement in terms of percentage of reduction in evidence of disease among subjects who have been administered a vaccine composition. For example, a vaccine efficacy of 50% indicates a 50% decrease in the number of disease cases among a group of vaccinated subjects as compared to a group of unvaccinated subjects or a group of subjects administered a different vaccine.

Wild type (WT): As is understood in the art, the term “wild type” generally refers to a normal form of a protein or nucleic acid, as is found in nature. For example, wild type HA and NA polypeptides are found in natural isolates of influenza virus. A variety of different wild type HA and NA sequences can be found in the NCBI influenza virus sequence database.

### Measuring Hemagglutinin Activity

Hemagglutinin activity may be measured using techniques known in the art, including, for example, hemagglutinin inhibition assay (HAI). An HAI applies the process of hemagglutination, in which sialic acid receptors on the surface of red blood cells (RBCs) bind to a hemagglutinin glycoprotein found on the surface of an influenza virus (and several other viruses) and create a network, or lattice structure, of interconnected RBCs and virus particles, referred to as hemagglutination, which occurs in a concentration dependent manner on the virus particles. This is a physical measurement taken as a proxy as to the facility of a virus to bind to similar sialic acid receptors on pathogen-targeted cells in the body. The introduction of anti-viral antibodies raised in a human or animal immune response to another virus (which may be genetically similar or different to the virus used to bind to the RBCs in the assay) interfere with the virus-RBC interaction and change the concentration of virus sufficient to alter the concentration at which hemagglutination is observed in the assay. One goal of an HAI can be to characterize the concentration of antibodies in the antiserum or other samples containing antibodies relative to their ability to elicit hemagglutination in the assay. The highest dilution of antibody that prevents hemagglutination is called the HAI titer (i.e., the measured response).

Another approach to measuring a HA antibody response is to measure a potentially larger set of antibodies elicited by a human or animal immune response, which are not necessarily capable of affecting hemagglutination in the HAI assay. A common approach for this leverages enzyme-linked immunosorbent assay (ELISA) techniques, in which a viral antigen (e.g., hemagglutinin) is immobilized to a solid surface, and then antibodies from the antisera are allowed to bind to the antigen. The readout measures the catalysis of a substrate of an exogenous enzyme complexed to either the antibodies from the antisera, or to other antibodies which themselves bind to the antibodies of the antisera. Catalysis of the substrate gives rise to easily detectable products. There are many variations of this sort of in vitro assay. One such variation is called antibody forensics (AF), which is a multiplexed bead array technique that allowed a single sample of serum to be measured against many antigens simultaneously. These measurements characterize the concentration and total antibody recognition, as compared to HAI titers, which are taken to be more specifically related to interference with sialic acid binding by hemagglutinin molecules. Therefore, an antisera's antibodies may in some cases have proportionally higher or lower measurements than the corresponding HAI titer for one virus's hemagglutinin molecules relative to another virus's hemagglutinin molecules; in other words, these two measurements, AF and HAI, may not be linearly related.

Another method of measuring HA antibody response includes a viral neutralization assay (e.g., microneutralization assay), wherein an antibody titer is measured by a reduction in plaques, foci, and/or fluorescent signal, depending on the specific neutralization assay technique, in permissive cultured cells following incubation of virus with serial dilutions of an antibody/serum sample.

### Measuring Neuraminidase Activity

Neuraminidase activity can be measured using techniques known in the art, including, for example, a MUNANA assay, ELLA assay, or an NA-Star® assay (ThermoFisher Scientific, Waltham, MA). In the MUNANA assay, 2′-(4-methylumbelliferyl)-alpha-D-N-acetylneuraminic acid (MUNANA) is used as a substrate. Any enzymatically active neuraminidase contained in the sample cleaves the MUNANA substrate, releasing 4-Methylumbelliferone (4-MU), a fluorescent compound. Thus, the amount of neuraminidase activity in a test sample correlates with the amount of 4-MU released, which can be measured using the fluorescence intensity (RFU, Relative Fluorescence Unit).

For purposes of determining the neuraminidase activity of a soluble tetrameric NA of the present disclosure, a MUNANA assay should be performed using the following conditions: mix soluble tetrameric NA with buffer [33.3 mM 2-(N-morpholino) ethanesulfonic acid (MES, pH 6.5), 4 mM CaCl2), 50 mM BSA] and substrate (100 μM MUNANA) and incubate for 1 hour at 37° C. with shaking; stop the reaction by adding an alkaline pH solution (0.2M Na2CO3); measure fluorescence intensity, using excitation and emission wavelengths of 355 and 460 nm, respectively; and calculate enzymatic activity against a 4MU reference. If necessary, an equivalent assay can be used to measure neuraminidase enzymatic activity.

### Vaccine Compositions

In certain aspects, disclosed herein is a vaccine composition comprising a plurality of generated amino acid sequences.

Each generated amino acid sequence may be present in the compositions disclosed herein in an amount effective to induce an immune response in a subject to which the composition is administered. In certain embodiments, each generated amino acid sequence may be present in the vaccine compositions disclosed herein in an amount ranging, for example, from about 0.1 g to about 500 g, such as from about 5 g to about 120 g, from about 1 g to about 60 g, from about 10 g to about 60 g, from about 15 g to about 60 g, from about 40 g to about 50 g, from about 42 g to about 47 g, from about 5 g to about 45 g, from about 15 g to about 45 g, from about 0.1 g to about 90 g, from about 5 g to about 90 g, from about 10 g to about 90 g, or from about 15 g to about 90 g. In certain embodiments, each recombinant HA may be present in the vaccine compositions disclosed herein in an amount of about 5 g, 10 g, 15 g, 20 g, 25 g, 30 g, 35 g, 40 g, 45 g, 50 g, 55 g, 60 g, 65 g, 70 g, 75 g, 80 g, 85 g, or about 90 g.

The vaccine composition can also further comprise an adjuvant. As used herein, the term “adjuvant” refers to a substance or vehicle that non-specifically enhances the immune response to an antigen. Adjuvants can include a suspension of minerals (alum, aluminum salts, including, for example, aluminum hydroxide/oxyhydroxide (AlOOH), aluminum phosphate (AlPO4), aluminum hydroxyphosphate sulfate (AAHS) and/or potassium aluminum sulfate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants also include biological molecules, such as lipids and costimulatory molecules. Exemplary biological adjuvants include AS04 (Didierlaurent, A. M. et al, AS04, an Aluminum Salt- and TLR4 Agonist-Based Adjuvant System, Induces a Transient Localized Innate Immune Response Leading to Enhanced Adaptive Immunity, J. IMMUNOL. 2009, 183: 6186-6197), IL-2, RANTES, GM-CSF, TNF-?, IFN-?, G-CSF, LFA-3, CD72, B7-1, B7-2, OX-40L and 41 BBL.

In certain embodiments, the adjuvant is a squalene-based adjuvant comprising an oil-in-water adjuvant emulsion comprising at least: squalene, an aqueous solvent, a polyoxyethylene alkyl ether hydrophilic nonionic surfactant, and a hydrophobic nonionic surfactant. In certain embodiments, the emulsion is thermoreversible, optionally wherein 90% of the population by volume of the oil drops has a size less than 200 nm.

In certain embodiments, the polyoxyethylene alkyl ether is of formula CH3-(CH2)x-(O—CH2-CH2)n-OH, in which n is an integer from 10 to 60, and x is an integer from 11 to 17. In certain embodiments, the polyoxyethylene alkyl ether surfactant is polyoxyethylene(12) cetostearyl ether.

In certain embodiments, 90% of the population by volume of the oil drops has a size less than 160 nm. In certain embodiments, 90% of the population by volume of the oil drops has a size less than 150 nm. In certain embodiments, 50% of the population by volume of the oil drops has a size less than 100 nm. In certain embodiments, 50% of the population by volume of the oil drops has a size less than 90 nm.

In certain embodiments, the adjuvant further comprises at least one alditol, including, but not limited to, glycerol, erythritol, xylitol, sorbitol and mannitol.

In certain embodiments the hydrophilic/lipophilic balance (HLB) of the hydrophilic nonionic surfactant is greater than or equal to 10. In certain embodiments, the HLB of the hydrophobic nonionic surfactant is less than 9. In certain embodiments, the HLB of the hydrophilic nonionic surfactant is greater than or equal to 10 and the HLB of the hydrophobic nonionic surfactant is less than 9.

In certain embodiments, the hydrophobic nonionic surfactant is a sorbitan ester, such as sorbitan monooleate, or a mannide ester surfactant. In certain embodiments, the amount of squalene is between 5 and 45%. In certain embodiments, the amount of polyoxyethylene alkyl ether surfactant is between 0.9 and 9%. In certain embodiments, the amount of hydrophobic nonionic surfactant is between 0.7 and 7%. In certain embodiments, the adjuvant comprises: i) 32.5% of squalene, ii) 6.18% of polyoxyethylene(12) cetostearyl ether, iii) 4.82% of sorbitan monooleate, and iv) 6% of mannitol.

In certain embodiments, the adjuvant further comprises an alkylpolyglycoside and/or a cryoprotective agent, such as a sugar, in particular dodecylmaltoside and/or sucrose.

In certain embodiments, the adjuvant comprises AF03, as described in Klucker et al., AF03, an alternative squalene emulsion-based vaccine adjuvant prepared by a phase inversion temperature method, J. PHARM. SCI. 2012, 101(12):4490-4500, which is hereby incorporated by reference in its entirety. In certain embodiments, the adjuvant comprises a liposome-based adjuvant, such as SPA14. SPA14 is a liposome-based adjuvant (AS01-like) containing a toll-like receptor 4 (TLR4) agonist (E6020) and saponin (QS21).

In addition to the recombinant HAs, recombinant NAs, and optional adjuvant, the vaccine composition may also further comprise one or more pharmaceutically acceptable excipients. In general, the nature of the excipient will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, vaccine compositions to be administered can contain minor amounts of non-toxic auxiliary substances, such as wetting or emulsifying agents, pharmaceutically acceptable salts to adjust the osmotic pressure, preservatives, stabilizers, buffers, sugars, amino acids, and pH buffering agents and the like, for example sodium acetate or sorbitan monolaurate.

Typically, the vaccine composition is a sterile, liquid solution formulated for parenteral administration, such as intravenous, subcutaneous, intraperitoneal, intradermal, or intramuscular. The vaccine composition may also be formulated for intranasal or inhalation administration. The vaccine composition can also be formulated for any other intended route of administration.

In some embodiments, a vaccine composition is formulated for intradermal injection, intranasal administration or intramuscular injection. In some embodiments, injectables are prepared in conventional forms, either as liquid solutions or suspensions, solid forms suitable for solution or suspension in liquid prior to injection, or as emulsions. In some embodiments, injection solutions and suspensions are prepared from sterile powders or granules. General considerations in the formulation and manufacture of pharmaceutical agents for administration by these routes may be found, for example, in Remington's Pharmaceutical Sciences, 19th ed., Mack Publishing Co., Easton, PA, 1995; incorporated herein by reference. At present the oral or nasal spray or aerosol route (e.g., by inhalation) are most commonly used to deliver therapeutic agents directly to the lungs and respiratory system. In some embodiments, the vaccine composition is administered using a device that delivers a metered dosage of the vaccine composition. Suitable devices for use in delivering intradermal pharmaceutical compositions described herein include short needle devices such as those described in U.S. Pat. Nos. 4,886,499, 5,190,521, 5,328,483, 5,527,288, 4,270,537, 5,015,235, 5,141,496, 5,417,662 (all of which are incorporated herein by reference). Intradermal compositions may also be administered by devices which limit the effective penetration length of a needle into the skin, such as those described in WO 1999/34850, incorporated herein by reference, and functional equivalents thereof. Also suitable are jet injection devices which deliver liquid vaccines to the dermis via a liquid jet injector or via a needle which pierces the stratum corneum and produces a jet which reaches the dermis. Jet injection devices are described for example in U.S. Pat. Nos. 5,480,381, 5,599,302, 5,334,144, 5,993,412, 5,649,912, 5,569,189, 5,704,911, 5,383,851, 5,893,397, 5,466,220, 5,339,163, 5,312,335, 5,503,627, 5,064,413, 5,520,639, 4,596,556, 4,790,824, 4,941,880, 4,940,460, WO 1997/37705, and WO 1997/13537 (all of which are incorporated herein by reference). Also suitable are ballistic powder/particle delivery devices which use compressed gas to accelerate vaccine in powder form through the outer layers of the skin to the dermis. Additionally, conventional syringes may be used in the classical mantoux method of intradermal administration.

Preparations for parenteral administration typically include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

### Kits

Further disclosed herein are kits for the vaccine compositions as disclosed herein. Kits may include a suitable container comprising the vaccine composition or a plurality of containers comprising different components of the vaccine composition, optionally with instructions for use.

In certain embodiments, the kit may comprise a plurality of containers, including, for example, a first container comprising one or more isolated nucleic acids, peptides and/or proteins as disclosed herein.

### Nucleic Acids, Cloning, and Expression Systems

The present disclosure further provides artificial nucleic acid molecules. The nucleic acids may comprise DNA or RNA and may be wholly or partially synthetic or recombinant. Reference to a nucleotide sequence as set out herein encompasses a DNA molecule with the specified sequence and encompasses an RNA molecule with the specified sequence in which U is substituted for T, or a derivative thereof, such as pseudouridine, unless context requires otherwise. Other nucleotide derivatives or modified nucleotides can be incorporated into the artificial nucleic acid molecules.

The present disclosure also provides constructs in the form of a vector (e.g., plasmids, phagemids, cosmids, transcription or expression cassettes, artificial chromosomes, etc.) comprising an artificial nucleic acid molecule encoding the generated amino acid sequences as disclosed herein. The disclosure further provides a host cell which comprises one or more constructs as above.

Also provided are methods of making the isolated peptides and/or proteins using recombinant techniques known in the art and as discussed above. The production and expression of recombinant proteins is well known in the art and can be carried out using conventional procedures, such as those disclosed in Sambrook et al., Molecular Cloning: A Laboratory Manual (4th Ed. 2012), Cold Spring Harbor Press. For example, expression of the HA or NA polypeptide may be achieved by culturing under appropriate conditions host cells containing the artificial nucleic acid molecule encoding the HA or NA as disclosed herein. For example, expression of the recombinant HA or NA polypeptide may be achieved by culturing under appropriate conditions host cells containing the nucleic acid molecule encoding the HA or NA as disclosed herein. Following production by expression, the HA or NA may be isolated and/or purified using any suitable technique, then used as appropriate.

Systems for cloning and expression of a polypeptide in a variety of different host cells are well known in the art. Any protein expression system (e.g., stable or transient) compatible with the constructs disclosed in this application may be used to produce the generated amino acid sequences described herein.

Suitable vectors can be chosen or constructed, so that they contain appropriate regulatory sequences, including promoter sequences, terminator sequences, polyadenylation sequences, enhancer sequences, marker genes and other sequences as appropriate.

For expressing the generated amino acid sequences as disclosed herein, nucleic acids encoding the generated amino acid sequences can be introduced into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., vaccinia or, for insect cells, baculovirus. For bacterial cells, suitable techniques may include calcium chloride transformation, electroporation and transfection using bacteriophage. These techniques are well known in the art. (See, e.g., “Current Protocols in Molecular Biology,” Ausubel et al. eds., John Wiley & Sons, 2010). DNA introduction may be followed by a selection method (e.g., antibiotic resistance) to select cells that contain the vector.

The host cell may be a plant cell, a yeast cell, or an animal cell. Animal cells encompass invertebrate (e.g., insect cells), non-mammalian vertebrate (e.g., avian, reptile and amphibian) and mammalian cells. In one embodiment, the host cell is a mammalian cell. Examples of mammalian cells include, but are not limited to COS-7 cells, HEK293 cells; baby hamster kidney (BHK) cells; Chinese hamster ovary (CHO) cells; mouse sertoli cells; African green monkey kidney cells (VERO-76); human cervical carcinoma cells (e.g., HeLa); canine kidney cells (e.g., MDCK), and the like. In one embodiment, the host cells are CHO cells. In one embodiment, the host cells are insect cells.

### Methods of Use

The present disclosure provides methods of administering the vaccine compositions described herein to a subject. The methods may be used to vaccinate a subject against a virus (e.g., an influenza virus). In some embodiments, the vaccination method comprises administering to a subject in need thereof a vaccine composition comprising one or more isolated nucleic acids, peptides and/or proteins encoding the generated amino acid sequences as described herein (e.g., recombinant influenza virus Has as described herein or recombinant influenza virus NAs as described herein), and an optional adjuvant in an amount effective to vaccinate the subject against a virus (e.g., an influenza virus). Likewise, the present disclosure provides a vaccine composition comprising one or more isolated nucleic acids, peptides and/or proteins encoding the generated amino acid sequences described herein (e.g., influenza virus Has or NAs as described herein), and an optional adjuvant, for use in (or for the manufacture of a medicament for use in) vaccinating a subject against a virus (e.g., an influenza virus).

The present disclosure also provides methods of immunizing a subject against a virus (e.g., an influenza virus), comprising administering to the subject an immunologically effective amount of a vaccine composition comprising one or more recombinant influenza virus HAs or NAs as described herein, and an optional adjuvant.

In some embodiments, the method or use prevents a viral infection (e.g., an influenza virus infection) or disease in the subject. In some embodiments, the method or use raises a protective immune response in the subject. In some embodiments, the protective immune response is an antibody response.

The methods/use of immunizing provided herein can elicit a broadly neutralizing immune response against one or more viruses (e.g., influenza viruses). Accordingly, in various embodiments, the composition described herein can offer broad cross-protection against different types of viruses (e.g., influenza viruses). In some embodiments, the composition offers cross-protection against avian, swine, seasonal, and/or pandemic influenza viruses. In some embodiments, the methods/use of immunizing are capable of eliciting an improved immune response against one or more seasonal influenza strains (e.g., a standard of care strain). For example, the improved immune response may be an improved humoral immune response. In some embodiments, the methods/use of immunizing are capable of eliciting an improved immune response against one or more pandemic influenza strains. In some embodiments, the methods of immunizing are capable of eliciting an improved immune response against one or more swine influenza strains. In some embodiments, the methods/use of immunizing are capable of eliciting an improved immune response against one or more avian influenza strains.

In certain embodiments, provided herein are methods of enhancing or broadening a protective immune response in a subject, the method comprising administering to the subject an immunologically effective amount of the vaccine composition disclosed herein, wherein the vaccine composition increases the vaccine efficacy of a standard of care influenza virus vaccine composition by an amount ranging from about 5% to about 100%, such as from about 10% to about 25%, from about 20% to about 100%, from about 15% to about 75%, from about 15% to about 50%, from about 20% to about 75%, from about 20% to about 50%, or from about 40% to about 80%, such as about 40% to about 60% or about 60% to about 80%. In certain embodiments, the vaccine composition disclosed herein has a vaccine efficacy that is at least 5% greater than the vaccine efficacy of a standard of care influenza virus vaccine, such as a vaccine efficacy that is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than the vaccine efficacy of a standard of care influenza virus vaccine. Likewise, the present disclosure provides any of the vaccine compositions described herein for use in (or for the manufacture of a medicament for use in) enhancing or broadening a protective immune response in a subject.

Also provided are methods of preventing a viral disease (e.g., an influenza virus disease) in a subject, comprising administering to the subject a vaccine composition comprising one or more isolated nucleic acids, peptides and/or proteins encoding the generated amino acid sequences (e.g., recombinant influenza virus HAs or NAs as described herein), and an optional adjuvant in an amount effective to prevent a viral disease (e.g., an influenza virus disease) in the subject. Likewise, the present disclosure provides a vaccine composition comprising one or more recombinant influenza virus HAs or NAs as described herein, and an optional adjuvant, for use in (or for the manufacture of a medicament for use in) preventing a viral disease (e.g., an influenza virus disease) in a subject.

Also provided are methods of inducing an immune response against an influenza virus HA and an influenza virus NA in a subject, comprising administering to the subject a vaccine composition comprising one or more recombinant influenza virus HAs as described herein, one or more recombinant influenza virus NAs as described herein, and an optional adjuvant.

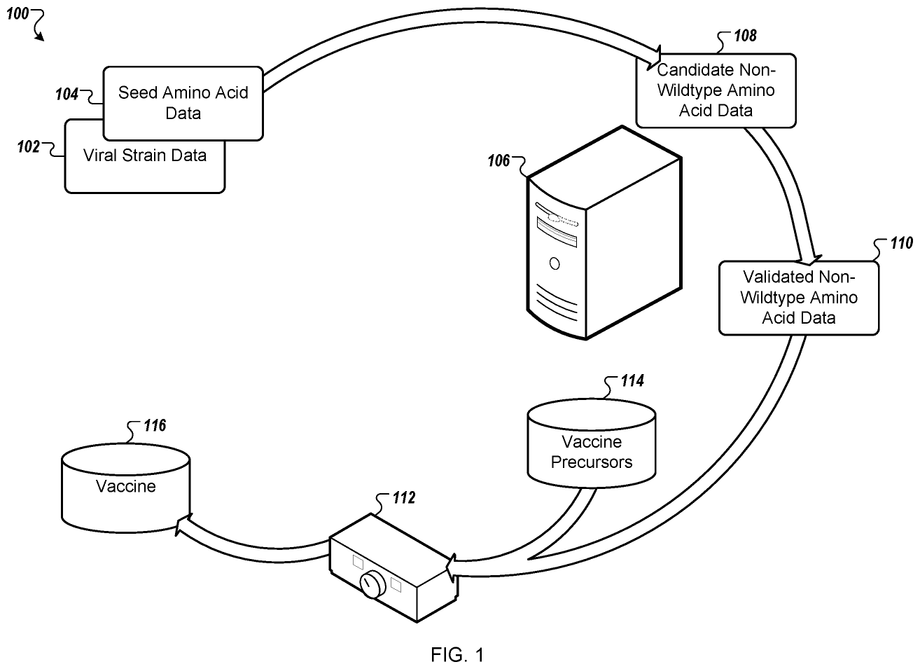

FIG. 1 is a block diagram of an example system 100 that can be used to manufacture a vaccine. In the system 100, a new vaccine 116 is designed and manufactured using technology described in this document. For example, for a virus with many strains, clades, serotypes, and/or strains that mutate quickly such as influenza or coronavirus disease 2019 or human rhinovirus, HIV, etc., or for new viruses never before encountered, the technology described here can be used to quickly generate vaccine candidates that can be tested for use in humans or other subjects.

As input, system 100 receives viral strain data 102, and seed amino acid data 104. Viral strain data 102 includes data about one or more viral strains against which vaccines are desired. This viral strain data 102 can include amino acid sequence data, as well as other types of data such as metadata (e.g., unique identifiers, strain identification) or non-metadata properties (e.g., records of physiochemical properties of the amino acid sequence such as molecular weight). Seed amino acid data 104 includes data about an initial or seed amino acid to be modified by a computer system 106 to generate a vaccine definition or definitions of candidate vaccines. This seed amino acid data 104 can include amino acid sequence data, as well as other types of data such as metadata (e.g., unique identifiers, strain identification) or non-metadata properties (e.g., records of physiochemical properties of the amino acid sequence such as molecular weight).

System 100 includes computer system 106 that can generate data 108 of candidate non-wildtype amino acid sequences by using the data 102 and 104. These non-wildtype amino acid sequences are sequences that are not found in the wild, or that are not known to be found in the wild. As will be appreciated, it is possible that one or more candidate non-wildtype amino acid sequences 108 may be in-fact in the wild, but not known to the operators of the system 100 or even to the community at large. The candidate non-wildtype amino acid sequence data 108 can include amino acid sequence data, as well as other types of data such as metadata (e.g., unique identifiers, strain identification) or non-metadata properties (e.g., records of physiochemical properties of the amino acid sequence such as molecular weight).

Computer system 106 validates one or more of the candidates in the data 108 for manufacture, resulting in data 110. The data 110 can include amino acid sequence data, as well as other types of data such as metadata (e.g., unique identifiers, strain identification) or non-metadata properties (e.g., records of physiochemical properties of the amino acid sequence such as molecular weight). In some cases, the data 102/104, 108, and 110 are in the same data format, and in some cases the data 102/104, 108, and 110 are in different data formats.

In some cases, the validation process used to select candidates can include determining if the amino acid sequence can be synthesized at all, or if it can be synthesized easily or economically. As will be appreciated, it is possible for an amino acid sequence to define a structure of a molecule that cannot exist in the physical world due to the geometry and forces such a molecule would exhibit. As such, such impossible sequences can be excluded from the validation process. In addition, some of the candidates may be excluded even though they define valid molecules. For example, the computer 106 can maintain a datastore of previous candidates that failed to actually be effective as a vaccine once investigated in clinical trials or predicted to be less immunogenic or less protective against viral strains of interest, which may include viral strain data 102. In such a case, candidates in the data 108 can be excluded from the validated data 110. In some cases, candidates can be excluded or prioritized based on synthetization and manufacturing considerations. For example, a candidate with particular synthesizing or handling conditions (e.g., cold storage, shock sensitivity) can be excluded from validation or deprioritized compared to other candidates having less onerous synthesizing or handling conditions.

System 100 can also include vaccine manufacturing devices 112 that can use vaccine precursors 114 and one or more validated non-wildtype amino acid sequence data 110 to manufacture one or more vaccine doses or vaccine molecules 116. As will be understood, initial exploration and testing would call for much smaller-scale synthesizing than large-scale manufacturing of a vaccine that has been tested, found safe and effective, and approved for use in humans or other subjects. Therefore, the particulars of the manufacturing devices 112 can vary according to the needs. Similarly, while the vaccine precursors 114 include those articles, chemicals, materials, etc. for the manufacture of the vaccine 116, the precursors 114 will similarly vary according to the needs.

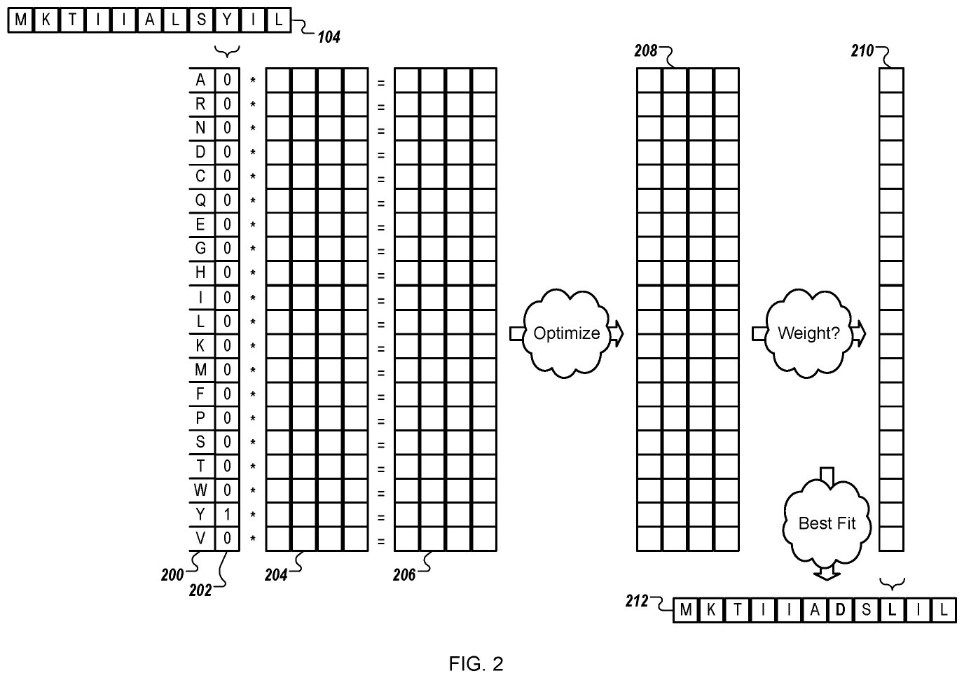

FIG. 2 is a schematic diagram of data that can be used in the manufacture of a vaccine. For example, the data shown here can be used by the computer system 106 or other computer systems.

Seed amino acid data 104 is shown here with a subsection of the sequence rendered for legibility using the single-letter designation recommended by the International Union of Pure and Applied Chemistry—International Union of Biochemistry and Molecular Biology (IUPAC-IUBMB) Joint Commission on Biochemical Nomenclature. The data 104 can include a vector of data values (e.g., single American Standard Code for Information Interchange (ASCII) characters, an integer) to represent the amino acids in the sequence represented by the data 104. As will be appreciated, longer sequences will have more indices than those shown visually here. In addition, other portions of the data 104 are not rendered here for clarity. This data 104 is a discrete-data object of one or more amino acid sequences. Each amino acid sequence can be recorded as either single letters or letter strings. The letter strings can include multiple single letters. The one or more amino acid sequences can include a first amino acid sequence and a second amino acid sequence, each of the first and the second amino acid sequences including respective single letters and respective letter strings. That is to say, each amino acid sequence can be stored in data that conforms to the same format, while holding different values. This can allow for interoperability and consistent handling of the data.

For each discrete value in the data 104, a corresponding weight-vector 202 can be created and maintained. The weight-vector 202 can be configured with an index for each possible amino acid in a particular index of the data 104 e.g., twenty weight values, each weight value corresponding to one of twenty possible amino acids 200. Initially, this probability can be set to either 0 or 1. As shown here, for an index with a value of “Y”, each index of the weight-vector 202 is set to zero, except for the second to last index location due to the fact that “Y” is the second to last possible amino acid label, when ordered alphabetically.

For each weight-value of the weight-vector 202, a corresponding property-vector 204 of property values is used. In this example, each property-vector 204 is a vector of length four, however other lengths are possible. Shown are twenty property-vectors 204, one for each of the twenty indices of the weight-vector 202. The weight-vector 202 and some subsequent vectors are shown without values, as they may be in formats (e.g., real numbers) that are too large to legibly render in the space provided.

In the property-vectors 204, each property value represents a physiochemical property of a particular amino acid. For example, the property-vectors may record the molecular weight, electrical charge, hydrophobic propensity, isoelectric point, alpha-helix propensity, beta-sheet propensity, molecular volume, octanol-water partition energy, etc.

Each value in the weight-vector 202 can be combined with the corresponding property-vector 204 to create a corresponding weighted-probability-vector 206. For example, each value in each property-vector 204 can be multiplied by the corresponding weight value in the weight-vector 202.

One or more optimization, solver, classifier, or other function can be applied to each weighted-probability-vector 206 (both those shown associated with the single index of vector 104, and all others associated with the other indices of vector 104), or to the set of weighted-probability-vectors 206, to generate an optimized-vector 208. Such functions will be described later in this document, but in general the function(s) can be configured to operate on continuous data (e.g., real numbers) to generate a second set of continuous data (e.g., real numbers) that more closely matches some target or property. As is shown, the optimized-vector 208 contains such continuous data while the data 104 instead contains discrete data (e.g., particular ASCII characters representing particular amino acids). The intermediate data 202-206 shown in FIG. 2 is used to process the data 104 for the functions that operate on continuous data. Once those functions are completed, the optimized-vector 208 can then be converted into discrete data for us in a real-world application such as specifying an amino acid sequence used to create a vaccine. While the term optimized is used here, it will be understood that this may or may not be an optimization in the strictest mathematical sense.

In the reverse of processes described, the optimized-vector 208 is used to find weights in a continuous-result-vector 210. These weights are values that, when multiplied by the corresponding property-vector 204, would produce the optimized vector 208. And while the weight-vector 202 contains only values of 0 and 1, the continuous-result-vector 210 is unlikely to have either the value 0 or 1, but instead continuous values between 0 and 1. Said another way, as value in a vector 206 changes to the value in 208, so would a value in vector 202 change to the value in vector 210.

A discrete-result object 212 is a found by finding, for each index, a best fit discrete value using the continuous-result-vector 210. As applied to the amino acid sequence example, this involves finding the amino acid at that location in the discrete-result object 212. This may include, for example, finding the greatest value in the continuous-result-vector 210 and selecting the amino acid that corresponds to that index location, though other best-fit processes are possible. Therefore the data 202-210 can be created for each index location in the vectors 104 and 212, thereby starting with discrete data 104, using one or more continuous-only functions, and generating discrete data 212. This advantageously allows for the transform of one amino acid sequence to another amino acid sequence, allowing for the synthesis and/or manufacture of new vaccines. A manufacturing device, e.g., 112, can use the discrete data 212 to generate vaccine precursors.

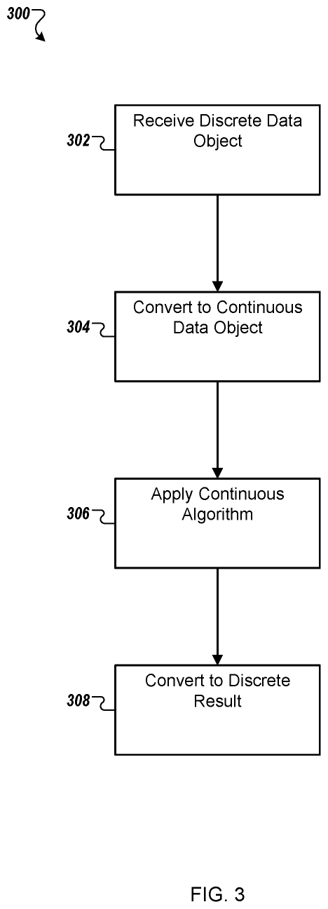

FIG. 3 is a flowchart of an example process 300 that can be used to apply continuous algorithms to discrete data, such as may be used in the manufacture of a vaccine. For example, the process 300 can be performed using the data shown in FIGS. 1 and 2, e.g., 102/104, 204-206, and will therefore use elements of those figures in the description. Possible embodiments of various elements of the process 300 are described later in processes 400-700.

A discrete-data object comprising a plurality of first discrete values is received 302. For example, a vector 104 representing seed amino acid sequence is received. This seed amino acid sequence may be, for example, a vaccine shown to be safe and effective against a previously encountered virus, an amino acid sequence previously observed in nature, an amino acid sequence not previously observed in nature but studied experimentally, a definition of a hypothetical molecule that would have desired properties but that cannot be or has not been yet synthesized, or randomly generated.

The discrete-data object includes one or more amino acid sequences. For example, the discrete-data object may take the form of binary data (i.e., l's and 0's) stored in computer memory and/or transmitted over a data network to the discrete/continuous converter 702. This binary data can be interpreted as a sequence of characters that specify one amino acid sequence or a group of amino acid sequences. In addition, other payload data (e.g., source of the sequence) and metadata (e.g., date of creation of the discrete-data object) may be included as well.

The discrete-data object is converted into a continuous-data object comprising a plurality of first continuous values 304. For example, the vector 104 can be converted into the vectors 206. This conversion process may or may not involve the use of vectors 202 and 204, depending on the particular processes used to perform this conversion.

A continuous-data algorithm is applied to the continuous-data object to generate a continuous-result object comprising a plurality of second continuous values 306. For example, one or more computer functions may be created based on mathematical or logical models that are designed to bring the amino acid sequence of vector 104 into a state more likely to have some property. One such example is to modify the amino acid sequence to create a new vaccine against an emerging virus or virus strain. This may include applying a gradient descent to the vectors 206 using a loss function that considers, among other parameters, how well a given amino acid sequence scores on a model predicting immune response given the viral strain data 102.

The continuous-result object is converted into a discrete-result object comprising a plurality of second discrete values 308. For example, the vectors 208 can be converted into the vector 212. This conversion process may or may not involve the use of vector 210, depending on the particular processes used to perform this conversion.

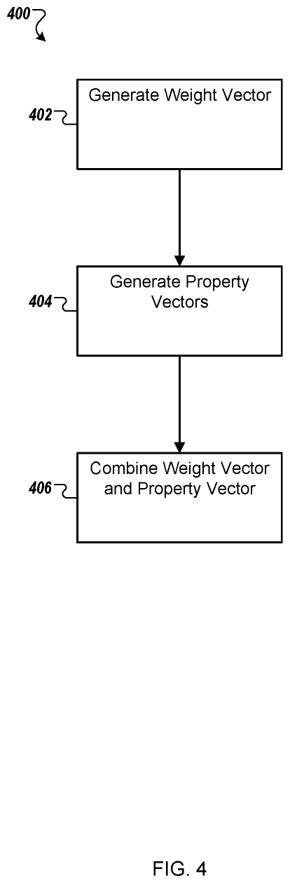

FIG. 4 is a flowchart of an example process that can be used to apply continuous algorithms to discrete data, such as may be used in the manufacture of a vaccine. For example, the process 400 can be performed using the data shown in FIGS. 1 and 2 and will therefore use elements of those figures in the description. The process 400 is a possible example of how operation 304 may be performed, though other processes may be used.

For each first discrete value, a weight-vector of weight values is generated, each weight value representing a likelihood that the first discrete value represents a particular amino acid 402. For example, for each value in the array 104, a corresponding vector 202 is generated. In this example, each value in the vector 104 is one specific amino acid, and therefore one and only one value in the vector 202 is a value of 1 while all other values are 0. However, another example may use a scheme where a location can have either one or another amino acid. In such a case, the associated vector 202 could have, for example, two values of 0.5.

For each weight value of each weight-vector, a property-vector of property values is generated, each property value representing a physiochemical property of a particular amino acid 404. For example, the vectors 204 may be accessed from a datastore that stores physiochemical or other properties of the various amino acids. These properties may be used in their original state, or may be preprocessed (e.g., normalized to be between 0 and 1, rounded to a given level of precision, converted to a different data format). As will be appreciated, these physiochemical or other properties may be recorded and held constant as they reflect observations and measurements of an amino acid, and these values may be available from a third party.

The weight-vector and the property-vector are combined to create a weighted-property-vector 406. For example, each weight value of the vector 202 can be multiplied by each corresponding vector 204 to create the vectors 206. These values may be post-processed (e.g., normalized to be between 0 and 1, rounded to a given level of precision, converted to a different data format).

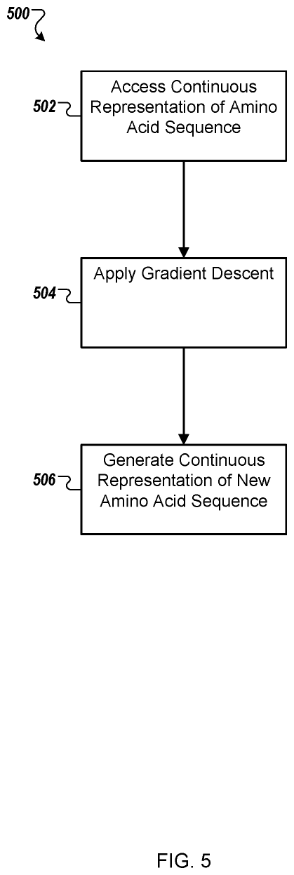

FIG. 5 is a flowchart of an example process that can be used to apply continuous algorithms to discrete data, such as may be used in the manufacture of a vaccine. For example, the process 500 can be performed using the data shown in FIGS. 1 and 2 and will therefore use elements of those figures in the description. The process 500 is a possible example of how operation 306 may be performed, though other processes may be used.

A continuous representation of an amino acid sequence is accessed 502. For example, for each index of the vector 104, twenty vectors 206 are accessed, resulting in a collection of vectors whose size is twenty times the length of the amino acid sequence represented by the vector 104.

A gradient descent is applied to the continuous representation 504. This gradient descent is configured with a loss function that determines a loss-value based on a plurality of loss criteria. Generally, the loss criteria can be conceptualized in two categories—the first to change the amino acid sequence toward a desired predicted property, including but not limited to, immunological response, and the second to scale-back to feasible amino acid sequences. If greater change to the original sequence is desired, the change-type can be applied first, followed by the scale-back-type. If less change to the original sequence is desired, a different order or intermingling of loss criteria may be used.

A first loss criteria is based on an immunological response given two amino acid sequences. For example, a predictor function may be configured to accept, as input, two amino acid sequences. This function may be configured to return, as output, a predicted immunity response of a subject (e.g., human, animal). This output may take the form of, for example, a value between 0 and 1, with higher values indicating a prediction of greater immunity response. This predictor function may operate using a machine-learning model.

A second loss criteria modifies (e.g., penalizes) sub-sequences not found in a dataset of wildtype sequences. For example, a datastore of known wildtype subsequences of amino acid sequences may be stored. If a given subsequence generated by this process is also found in wildtype amino acid sequence, it is likely to be a subsequence that can be synthesized. However, if a subsequence not found in any known wildtype amino acid sequence may be impossible to synthesize or may require the development of new synthetization to be possible or economical. Therefore, subsequences not found in wildtype amino acid sequences may be penalized to avoid these problems. In general, the second loss criteria may include a score resulting from a machine learning model.

A third loss criteria penalizes, for each weight-vector, the weight-vector based on the greatest value in the vector of weights. For example, if a weight in the vector 210 is near 1 (i.e. the maximum value), this is a high confidence rating or indication of high immune response for a given amino acid in a particular location in the sequence of the vector 212. In this case, the third loss criteria may apply no penalty or a small penalty. In another example, if the greatest weight value is much lower, this is a low confidence rating or indication of low immunity response and thus may have a larger penalty applied. In some cases, the penalty may be to multiply a score by a value of 1 minus the greatest weight, though other schemes may be used.

As will be appreciated, some, all, or none of these criteria may be used.

A continuous representation of a new amino acid sequence is generated 506. For example, for each index of the vector 104, which contains a discrete representation of a single amino acid, twenty vectors 208 may be created holding continuous values. As is described elsewhere in this document, these twenty vectors 208 can be converted into a single discrete value in the vector 212 to represent a single amino acid.

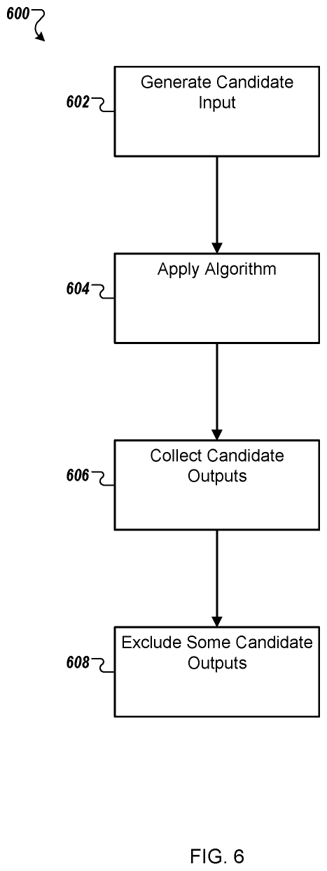

FIG. 6 is a flowchart of an example process that can be used to apply continuous algorithms to discrete data, such as may be used in the manufacture of a vaccine. For example, the process 600 can be performed using the data shown in FIGS. 1 and 2 and will therefore use elements of those figures in the description.

A plurality of candidate discrete-result objects are generated 602. For example, for a single viral strain data object 102, a large collection of seed amino acid data objects 104 can be created. This can include collecting all known viable vaccines for a given virus and using those as seed data 104 for a new strain of the virus 102.

For each seed, an algorithm to change the sequence is applied 604. For example, the process 300 can be performed using each seed to generate an equal number of candidate sequences.

The candidate outputs are collected 606 and some are excluded 608. For example, at least one candidate may be found to specify an amino acid sequence failing a manufacturability test. This test may involve determining that the sequence is impossible to synthesize, too similar to another candidate, a match for one of the seeds, etc. This can allow, for example, the most likely candidates to be prioritized when testing resources are limited.

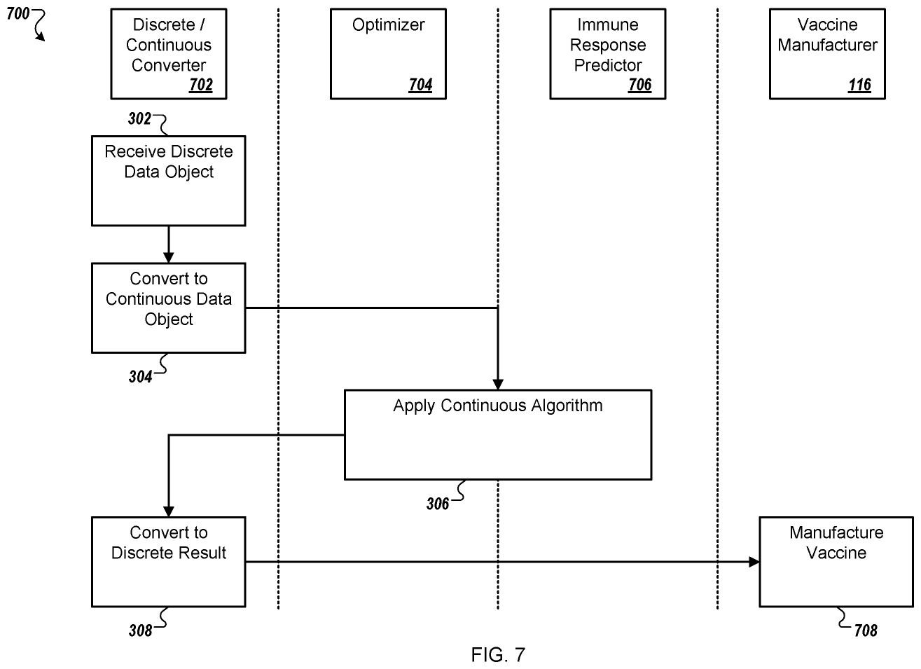

FIG. 7 is a swimlane diagram of an example process to manufacture a vaccine. For example, the process 400 can be performed using the data shown in FIGS. 1 and 2 and will therefore use elements of those figures in the description. The process 700 incorporates the process 300, and will therefore be shown with elements of the process 300. To perform the process 700, the computer system 106 can use a discrete/continuous converter 702, an optimizer 704, and an immune response predictor 706, though different components may be used.

Once data defining an amino acid sequence to be manufactured is generated, the vaccine manufacturer 116 manufactures 708 a vaccine comprising a protein defined by the discrete-result object (i.e. the amino acid sequence) and/or a vaccine comprising a nucleic acid, or any other delivery vehicle including viral or bacterial vectors, whereby such nucleic acid or delivery vehicle produces the protein defined by the discrete-results object. This manufacture may be a small batch for purposes of initial test, for clinical trials, and/or for general use. As will be appreciated, the elements 308 and 708 may be separated by a significant amount of time and interstitial operations. For example, if the manufacturing in 708 is large-volume manufacture for general use, this may be only after clinical trials have demonstrated that the vaccine is safe and effective for its intended purpose.

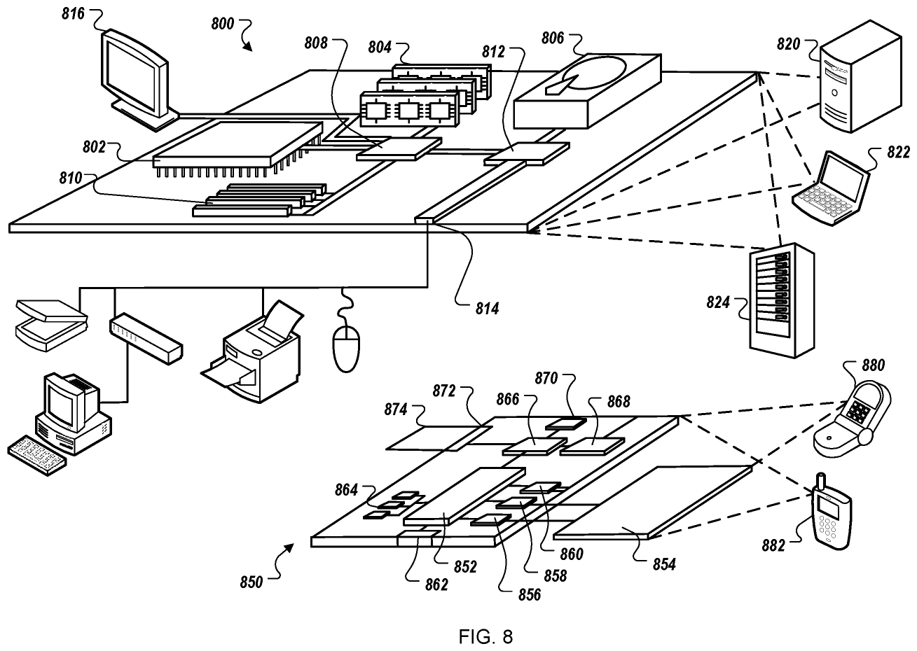

FIG. 8 shows an example of a computing device 800 and an example of a mobile computing device that can be used to implement the techniques described here. The computing device 800 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 800 includes a processor 802, a memory 804, a storage device 806, a high-speed interface 808 connecting to the memory 804 and multiple high-speed expansion ports 810, and a low-speed interface 812 connecting to a low-speed expansion port 814 and the storage device 806. Each of the processor 802, the memory 804, the storage device 806, the high-speed interface 808, the high-speed expansion ports 810, and the low-speed interface 812, are interconnected using various busses, and can be mounted on a common motherboard or in other manners as appropriate. The processor 802 can process instructions for execution within the computing device 800, including instructions stored in the memory 804 or on the storage device 806 to display graphical information for a GUI on an external input/output device, such as a display 816 coupled to the high-speed interface 808. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 804 stores information within the computing device 800. In some implementations, the memory 804 is a volatile memory unit or units. In some implementations, the memory 804 is a non-volatile memory unit or units. The memory 804 can also be another form of computer-readable medium, such as a magnetic or optical disk.

The storage device 806 is capable of providing mass storage for the computing device 800. In some implementations, the storage device 806 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 804, the storage device 806, or memory on the processor 802.

The high-speed interface 808 manages bandwidth-intensive operations for the computing device 800, while the low-speed interface 812 manages lower bandwidth-intensive operations. Such allocation of functions is exemplary only. In some implementations, the high-speed interface 808 is coupled to the memory 804, the display 816 (e.g., through a graphics processor or accelerator), and to the high-speed expansion ports 810, which can accept various expansion cards (not shown). In the implementation, the low-speed interface 812 is coupled to the storage device 806 and the low-speed expansion port 814. The low-speed expansion port 814, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 800 can be implemented in a number of different forms, as shown in the figure. For example, it can be implemented as a standard server 820, or multiple times in a group of such servers. In addition, it can be implemented in a personal computer such as a laptop computer 822. It can also be implemented as part of a rack server system 824. Alternatively, components from the computing device 800 can be combined with other components in a mobile device (not shown), such as a mobile computing device 850. Each of such devices can contain one or more of the computing device 800 and the mobile computing device 850, and an entire system can be made up of multiple computing devices communicating with each other.

The mobile computing device 850 includes a processor 852, a memory 864, an input/output device such as a display 854, a communication interface 866, and a transceiver 868, among other components. The mobile computing device 850 can also be provided with a storage device, such as a micro-drive or other device, to provide additional storage. Each of the processor 852, the memory 864, the display 854, the communication interface 866, and the transceiver 868, are interconnected using various buses, and several of the components can be mounted on a common motherboard or in other manners as appropriate.

The processor 852 can execute instructions within the mobile computing device 850, including instructions stored in the memory 864. The processor 852 can be implemented as a chipset of chips that include separate and multiple analog and digital processors. The processor 852 can provide, for example, for coordination of the other components of the mobile computing device 850, such as control of user interfaces, applications run by the mobile computing device 850, and wireless communication by the mobile computing device 850.

The processor 852 can communicate with a user through a control interface 858 and a display interface 856 coupled to the display 854. The display 854 can be, for example, a TFT (Thin-Film-Transistor Liquid Crystal Display) display or an OLED (Organic Light Emitting Diode) display, or other appropriate display technology. The display interface 856 can comprise appropriate circuitry for driving the display 854 to present graphical and other information to a user. The control interface 858 can receive commands from a user and convert them for submission to the processor 852. In addition, an external interface 862 can provide communication with the processor 852, so as to enable near area communication of the mobile computing device 850 with other devices. The external interface 862 can provide, for example, for wired communication in some implementations, or for wireless communication in other implementations, and multiple interfaces can also be used.

The memory 864 stores information within the mobile computing device 850. The memory 864 can be implemented as one or more of a computer-readable medium or media, a volatile memory unit or units, or a non-volatile memory unit or units. An expansion memory 874 can also be provided and connected to the mobile computing device 850 through an expansion interface 872, which can include, for example, a SIMM (Single In Line Memory Module) card interface. The expansion memory 874 can provide extra storage space for the mobile computing device 850, or can also store applications or other information for the mobile computing device 850. Specifically, the expansion memory 874 can include instructions to carry out or supplement the processes described above, and can include secure information also. Thus, for example, the expansion memory 874 can be provide as a security module for the mobile computing device 850, and can be programmed with instructions that permit secure use of the mobile computing device 850. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 864, the expansion memory 874, or memory on the processor 852. In some implementations, the computer program product can be received in a propagated signal, for example, over the transceiver 868 or the external interface 862.

The mobile computing device 850 can communicate wirelessly through the communication interface 866, which can include digital signal processing circuitry where necessary. The communication interface 866 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 868 using a radio-frequency. In addition, short-range communication can occur, such as using a Bluetooth, WiFi, or other such transceiver (not shown). In addition, a GPS (Global Positioning System) receiver module 870 can provide additional navigation- and location-related wireless data to the mobile computing device 850, which can be used as appropriate by applications running on the mobile computing device 850.

The mobile computing device 850 can also communicate audibly using an audio codec 860, which can receive spoken information from a user and convert it to usable digital information. The audio codec 860 can likewise generate audible sound for a user, such as through a speaker, e.g., in a handset of the mobile computing device 850. Such sound can include sound from voice telephone calls, can include recorded sound (e.g., voice messages, music files, etc.) and can also include sound generated by applications operating on the mobile computing device 850.

The mobile computing device 850 can be implemented in a number of different forms, as shown in the figure. For example, it can be implemented as a cellular telephone 880. It can also be implemented as part of a smart-phone 882, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

1. A method for manufacturing a vaccine by using a continuous-data algorithm, the method comprising:
receiving a discrete-data object comprising a plurality of first discrete values, the discrete-data object comprising one or more amino acid sequences;
converting the discrete-data object into a continuous-data object comprising a plurality of first continuous values;
applying, to the continuous-data object, a continuous-data algorithm to generate a continuous-result object comprising a plurality of second continuous values;
converting the continuous-result object into a discrete-result object comprising a plurality of second discrete values; and
manufacturing a vaccine comprising at least one of the group consisting of i) a protein defined by the discrete-result object, ii) a nucleic acid capable of producing the protein defined by the discrete-result object, and iii) a delivery vehicle capable of producing the protein defined by the discrete-result object.

2. The method of claim 1, wherein the one or more amino acid sequences comprises:
a first amino acid sequence and a second amino acid sequence, each of the first and the second amino acid sequences including respective single letters or respective letter strings.

3. The method of claim 1, wherein converting the discrete-data object into the continuous-data object comprises:
generating, for each first discrete value, a weight-vector of weight values, each weight value representing a likelihood that the first discrete value represents a particular amino acid;
generating, for each weight value of each weight-vector, a property-vector of property values, each property value representing a physiochemical property of a particular amino acid; and
combining the weight-vector and the property-vector to create the first continuous values of the continuous-data object.

4. The method of claim 3, wherein each weight-vector has twenty weight values, each weight value corresponding to one of twenty possible amino acids.

5. The method of claim 3, wherein converting the continuous-result object into the discrete-result object comprises determining, for each second continuous value, a respective single amino acid, wherein the determined single amino acids form the plurality of second discrete values.

6. The method of claim 3, wherein the method further comprises:
generating a plurality of candidate discrete-result objects; and
excluding, from the plurality of candidate discrete-result objects, at least one discrete-result object that specifies an amino acid failing a manufacturability test.

7. The method of claim 3, wherein applying the continuous-data algorithm to generate the continuous-result object comprises applying a gradient descent with a loss function that determines a loss-value based on a plurality of loss criteria, the loss function comprising:
a first loss criteria based on an immunological response given two amino acid sequences;
a second loss criteria that modifies the loss-value for sub-sequences not found in a dataset of wildtype sequences or sub-sequences not predicted to fold correctly; and
a third loss criteria that, for each weight-vector, modifies the loss-value based on the greatest value in the second continuous values.

8. The method of claim 1, wherein the vaccine is for one of the group consisting of i) influenza, ii) human rhinovirus, iii) HIV and iv) a coronavirus disease.

9. A system for generating amino acid sequences, the system comprising;
one or more processors; and
computer-memory storing instructions that, when executed by the processors, cause the processors to perform operations comprising:
receiving a discrete-data object comprising a plurality of first discrete values, the discrete-data object comprising one or more amino acid sequences;
converting the discrete-data object into a continuous-data object comprising a plurality of first continuous values;
applying, to the continuous-data object, a continuous-data algorithm to generate a continuous-result object comprising a plurality of second continuous values;
converting the continuous-result object into a discrete-result object comprising a plurality of second discrete values; and
manufacturing a vaccine comprising at least one of the group consisting of i) a protein defined by the discrete-result object, ii) a nucleic acid capable of producing the protein defined by the discrete-result object, and iii) a delivery vehicle capable of producing the protein defined by the discrete-result object.

10. The system of claim 9, wherein the one or more amino acid sequences comprises:
a first amino acid sequence and a second amino acid sequence, each of the first and the second amino acid sequences including respective single letters or respective letter strings.

11. The system of claim 9, wherein converting the discrete-data object into the continuous-data object comprises:
generating, for each first discrete value, a weight-vector of weight values, each weight value representing a likelihood that the first discrete value represents a particular amino acid;
generating, for each weight value of each weight-vector, a property-vector of property values, each property value representing a physiochemical property of a particular amino acid; and
combining the weight-vector and the property-vector to create the first continuous values of the continuous-data object.

12. The system of claim 11, wherein each weight-vector has twenty weight values, each weight value corresponding to one of twenty possible amino acids.

13. The system of claim 11, wherein converting the continuous-result object into the discrete-result object comprises determining, for each second continuous value, a respective single amino acid, wherein the determined single amino acids form the plurality of second discrete values.

14. The system of claim 11, wherein the operations further comprise:
generating a plurality of candidate discrete-result objects; and
excluding, from the plurality of candidate discrete-result objects, at least one discrete-result object that specifies an amino acid failing a manufacturability test.

15. The system of claim 11, wherein applying the continuous-data algorithm to generate the continuous-result object comprises applying a gradient descent with a loss function that determines a loss-value based on a plurality of loss criteria, the loss function comprising:
a first loss criteria based on an immunological response given two amino acid sequences;
a second loss criteria that modifies the loss-value for sub-sequences not found in a dataset of wildtype sequences or sub-sequences not predicted to fold correctly; and
a third loss criteria that, for each weight-vector, modifies the loss-value based on the greatest value in the second continuous values.

16. A non-transitory, computer readable media storing instructions that, when executed by one or more processors, cause the one or more processors to perform operations comprising:
receiving a discrete-data object comprising a plurality of first discrete values, the discrete-data object comprising one or more amino acid sequences;
converting the discrete-data object into a continuous-data object comprising a plurality of first continuous values;
applying, to the continuous-data object, a continuous-data algorithm to generate a continuous-result object comprising a plurality of second continuous values;
converting the continuous-result object into a discrete-result object comprising a plurality of second discrete values; and
manufacturing a vaccine comprising at least one of the group consisting of i) a protein defined by the discrete-result object, ii) a nucleic acid capable of producing the protein defined by the discrete-result object, and iii) a delivery vehicle capable of producing the protein defined by the discrete-result object.

17. The media of claim 16, wherein the one or more amino acid sequences comprises:
a first amino acid sequence and a second amino acid sequence, each of the first and the second amino acid sequences including respective single letters or respective letter strings.

18. The media of claim 16, wherein converting the discrete-data object into the continuous-data object comprises:
generating, for each first discrete value, a weight-vector of weight values, each weight value representing a likelihood that the first discrete value represents a particular amino acid;
generating, for each weight value of each weight-vector, a property-vector of property values, each property value representing a physiochemical property of a particular amino acid; and
combining the weight-vector and the property-vector to create the first continuous values of the continuous-data object.

19. The media of claim 18, wherein each weight-vector has twenty weight values, each weight value corresponding to one of twenty possible amino acids.

20. The media of claim 18, wherein converting the continuous-result object into the discrete-result object comprises determining, for each second continuous value, a respective single amino acid, wherein the determined single amino acids form the plurality of second discrete values.


SEQUENCE LISTING (1 sequence)

SEQ ID NO: 1 - protein, 566 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRGSSSSFFSRLNWLTHLNYTYPALNVTMPNKEQFDKLYIWGVHHPGTDKDQISLYARSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI